(19) United States
(12) Patent Application Publication
(10) Pub. No.: US 2021/0139925 A1
(43) Pub. Date: May 13, 2021

(54) MAIZE FEMALE PARENT HAPLOID MAJOR EFFECT INDUCING GENE AND APPLICATION

(71) Applicant: CHINA AGRICULTURAL UNIVERSITY, Beijing (CN)

(72) Inventors: Shaojiang CHEN, Beijing (CN); Chenxu LIU, Beijing (CN); Xin DONG, Beijing (CN); Xiaowei XU, Beijing (CN); Liang LI, Beijing (CN); Yu ZHONG, Beijing (CN); Chen CHEN, Beijing (CN)

(21) Appl. No.: 16/477,635

(22) PCT Filed: Jan. 13, 2017
(86) PCT No.: PCT/CN2017/071079

§ 371 Date: Jul. 12, 2019

(51) Int. Cl. C12N 15/82; C12N 15/113

(52) U.S. Cl. CPC C12N 15/8218; C12N 15/113

(57) ABSTRACT

The present invention discloses a maternal haploid major inducing gene in maize and application thereof and also discloses a major gene for inducing the production of maize maternal haploid and its application in double haploid (DH) breeding. This gene encodes a phospholipase (PLA) and the nucleotide sequence thereof is SEQ ID NO: 1. This gene has a maternal haploid inducing ability during selfing or the hybridization of the material as a paternal and other maize material, after a mutation occurred in the coding region. The present invention obtains a series of allelic mutations of the gene for the first time, and proves its maternal haploid induction function by selfing and hybridization. The acquisition of the maternal haploid has important theoretical and practical significance for haploid breeding (DH) and related research.

### INCORPORATION BY REFERENCE

The sequence listing provided in the file entitled C6351-009_Sequence_Listing_v3.txt, which is an ASCII text file that was created on Aug. 28, 2020, and which comprises 4096 bytes, is hereby incorporated by reference in its entirety.

### TECHNICAL FIELD

The present invention relates to the field of biotechnology, and particularly relates to a maternal haploid major inducing gene in maize and application thereof.

### BACKGROUND ART

Maize is the world's largest crop with a variety of uses in food, feed and industrial processing, etc. The increase in maize production is of great importance for the supply of current food, feed and industrial processing needs. In the current gradual reduction of cultivated land area, it is the key to cultivate maize hybrids with high yield, multi-resistance and wide adaptability. The breeding of maize hybrids depends on the selection of elite inbred lines. The traditional method of breeding inbred lines is time-consuming and laborious, and it is often necessary to pass through 7 generations to develop a stable inbred line. In recent years, haploid breeding technique has the advantages of short breeding cycle, high efficiency, easy to combine molecular marker-assisted breeding methods, etc., and have gradually become the main technology for breeding maize inbred lines. At present, haploids in maize are mainly derived from the induction of parthenogenetic inducing lines in maize, which are produced from the hybridization of Stock6 or its derived inducing line as a male parent with other materials. Since most of the inducing lines are introduced with the R1-nj marker, the identification of maize haploids can be performed using embryo and endosperm color markers. Therefore, the efficiency of the maize haploid breeding is greatly improved.

Since the method of producing maternal haploids via the production of parthenogenesis by inducing lines has broad application prospects and value, many research institutes in the world have conducted extensive research on the genetic basis and biological basis of Stock6 and its derived lines to induce maternal haploids. The results showed that the trait of maize parthenogenesis induction to produce maize haploid is heritable and controlled by multiple genetic loci. Röber (1999) et al. detected two genetic loci that control the trait of induction rate, which are located on chromosome 1 and chromosome 2, respectively and can explain about 17% of phenotypic variation. Barrant et al. (2008) also detected the genetic locus on chromosome 1, validating the results of previous studies. Prigge et al. (2012) used multiple populations for whole-genome scan and a total of eight genetic loci controlling the induction rate were found, including the major genetic locus at 1.04 bin on chromosome 1, and named ghir1. Therefore, ghir1 is a QTL with the maximum effects and most important functions in multiple QTLs related to the control of haploid induction rate. Dong Xin et al. (2014) conducted a fine mapping of qhir1 and successfully narrowed the interval to a range of 243 Kb. It is particularly important to study the candidate genes of ghir1 for the selection of novel inducing lines and the genetic and biological mechanisms of parthenogenetic inducing lines inducing haploids. In view of the extensive use of haploid breeding technique in the breeding industry, the present invention has a very wide application space and market prospects.

### SUMMARY OF THE INVENTION

It is an object of the present invention to provide a maternal haploid major inducing gene in maize, ZmPLA mutant gene.

The ZmPLA mutant gene provided by the present invention has a nucleotide sequence which is obtained by conducting insertion or/and deletion or/and substitution mutations in the nucleotide sequence of the wild-type ZmPLA gene;

the nucleotide sequence of the wild-type ZmPLA gene is SEQ ID NO: 1.

In the above gene, the nucleotide sequence of the ZmPLA mutant gene is any one of the following 1) to 4) (corresponding to the ZmPLA mutant genes ZmHIR1-1, ZmHIR1-2, ZmHIR1-3 and ZmHIR1-Stock6 in the Examples below):

1) a sequence obtained by inserting a base T between positions 280 and 281 of the nucleotide sequence of the wild-type ZmPLA gene and keeping the other bases unchanged;
2) a sequence obtained by deleting the bases at positions 271-281 of the nucleotide sequence of the wild-type ZmPLA gene and keeping the other bases unchanged;
3) a sequence obtained by deleting the base G at position 2810f the nucleotide sequence of the wild-type ZmPLA gene and keeping the other bases unchanged;
4) a DNA molecule with a sequence obtained by inserting CGAG after position 1569 of the nucleotide sequence of the wild-type ZmPLA gene, and mutating the base C at position 409 to a base T, mutating the base C at position 421 to a base G, mutating the base T at position 44 to a base C, mutating the base T at position 887 to a base G, mutating the base G at position 1210 to a base C, mutating the base T at position 1306 to a base C, mutating the base G at position 1435 to a base A, mutating the base C at position 1471 to a base A, mutating the base A at position 1541 to a base C, mutating the base T at position 1588 to a base C and mutating the base C at position 1591 to a base A, mutating the base A at position 1687 to a base C, mutating the base G at position 1691 to a base A, mutating the base T at position 1706 to a base C, mutating the base G at position 1708 to a base C, deleting the two bases TA at positions 45-46, substituting the bases TCG at positions 65-67 with bases CAA, inserting two bases TC between positions 67 and 68, substituting the bases TT at positions 80-81 with bases CG, deleting the bases GTAC at positions 499-503, mutating the base C at position 524 to a base G, mutating the base G at position 530 to a base T, deleting the bases GCATGCAT at positions 553-560, deleting the bases GTAC at positions 806-809, mutating the base G at position 1741 to a base A, mutating the base C at position 1781 to a base T, mutating the base A at position 1787 to a base T and keeping the other bases unchanged.

Use of the above-described mutant gene or the nucleotide sequence of the wild-type ZmPLA gene for inducing the production of maize or other plant haploid or in double haploid (DH) breeding is also within the protection scope of the present invention.

Use of silencing or inhibiting the expression of the ZmPLA gene or knocking out the ZmPLA gene in the genome of a target plant in the production of a plant haploid is also within the protection scope of the present invention; or use of a substance which silences or inhibits the expression of the ZmPLA gene or knocks out the ZmPLA gene in the genome of a target plant in the production of a plant maternal haploid is also within the protection scope of the present invention.

The above use is to silence or inhibit or knock out the expression of the ZmPLA gene in the genome of the target plant to obtain a transgenic plant, and then use the transgenic plant for hybridization or selfing to obtain a maternal haploid.

In the above uses, the silencing or inhibiting the expression of the ZmPLA gene in the genome of the target plant or knocking out the ZmPLA gene is such that the expression level of the ZmPLA gene in the genome of the target plant is decreased or a deletion or insertion mutation occurs;

In the above uses, the deletion or insertion mutation in the ZmPLA gene in the genome of the target plant is a deletion or insertion mutation in the first exon and/or the second exon and/or the third exon and/or the fourth exon of the ZmPLA gene in the genome of the target plant; or

a way to cause the deletion or insertion mutation in the ZmPLA gene in the genome of the target plant is CRISPR/Cas9 and/or TALEN technology and/or T-DNA insertion and/or EMS mutagenesis.

In the above uses, the way to cause the deletion or insertion mutation in the first exon of the ZmPLA gene in the genome of the target plant is CRISPR/Cas9; or

the substance which silences or inhibits the expression of the ZmPLA gene or knocks out the ZmPLA gene in the genome of the target plant is a substance which causes a deletion or insertion mutation in the first exon of the ZmPLA gene in the genome of the target plant;
the substance which causes deletion or insertion mutation in the first exon of the ZmPLA gene in the genome of the target plant is CRISPR/Cas9 system;
the target sequence of the CRISPR/Cas9 system is positions 264-286 of the first exon and shown in SEQ ID NO: 3;
the sgRNA sequence of the CRISPR/Cas9 system is SEQ ID NO: 4.

Use of silencing or inhibiting the expression of the ZmPLA gene or knocking out the ZmPLA gene in the genome of a target plant in double haploid (DH) breeding or DH line-based hybrid breeding is also within the protection scope of the present invention. Or use of a substance which silences or inhibits the expression of the ZmPLA gene or knocks out the ZmPLA gene in the genome of a target plant in double haploid (DH) breeding or DH line-based hybrid breeding is also within the protection scope of the present invention.

The above target plants are maize or other plants.

Another object of the present invention is to provide a substance which silences or inhibits the expression of the ZmPLA gene or knocks out the ZmPLA gene in the genome of a target plant.

The substance provided by the present invention comprises a CRISPR/Cas9 system, wherein the target sequence of the CRISPR/Cas9 system is positions 264-286 of the first exon and shown in SEQ ID NO: 3.

In the above substance, the sgRNA sequence of the CRISPR/Cas9 system is SEQ ID NO: 4.

The technical solution of the present invention is as follows: through a candidate gene prediction, a gene encoding phospholipase (PLA) is obtained in the ghir1 interval and named ZmPLA and a mutant material of the target gene is successfully obtained by the CRISPR/Cas9 site-directed mutagenesis technique and the transgenic assay, a heterozygous genotype mutant and a homozygous genotype mutant are used to hybridize with other maize materials, it is verified that the ZmPLA mutant material can be used as a male parent to induce maternal haploid, and the ZmPLA gene that has no function after sequence mutation is named ZmHIR. The artificial site-directed mutagenesis of the ZmPLA gene uses the CRISPR/Cas9 site-directed mutagenesis technique to modify the first exon of the ZmPLA gene such that the bases of the first exon are replaced, deleted and/or inserted. The modified target of the CRISPR/Cas9 has a designed length of 20 bp and is located at positions 264-286 of the first exon of ZmPLA and the sequence of the target site is: GCTGCAGGAGCTGGACGGACCGG.

The ZmPLA artificial site-directed mutant produced by the CRISPR/Cas9 site-directed mutagenesis technique at the target site is characterized in that the CRISPR/Cas9 gene modification technique causes a 1 bp T base insertion between positions 280 and 281 at the modification target site to obtain a ZmPLA gene mutant, and the first exon sequence after insertion of the base, the gene after the insertion of the base is named ZmHIR1-1, and the progeny of this mutant can produce about 1%-2% of maize maternal haploid.

The ZmPLA artificial site-directed mutant produced by the CRISPR/Cas9 site-directed mutagenesis technique at the target site is characterized in that the CRISPR/Cas9 gene modification technique causes the deletion of GAGCTGGACGG at positions 271-281 at the modification target site to obtain a ZmPLA gene mutant, and the first exon sequence after the deletion of the bases, the gene after the deletion of the bases is named ZmHIR1-2, and the progeny of this mutant can produce about 1%-2% of maize maternal haploid.

The ZmPLA artificial site-directed mutant produced by the CRISPR/Cas9 site-directed mutagenesis technique at the target site is characterized in that the CRISPR/Cas9 gene modification technique causes the deletion of the base G at position 281 at the modification target site to obtain a ZmPLA gene mutant, and the first exon sequence after the deletion of the base, the gene after the deletion of the base is named ZmHR1-3, and the progeny of this mutant can produce about 1%-2% of maize maternal haploid.

The present invention also provides a mutant gene sequence of a known maize maternal haploid inducing line Stock6, and names it ZmHIR1-Stock6, characterized by ZmHIR1-Stock6 resulting in that haploid appears in the progeny of the selfing or hybridizing as a male parent with other materials. The sequence is obtained by the candidate gene prediction and sequencing of the present invention and the loss of the function of the gene is confirmed by transgenic experiments to cause the production of the maize maternal haploid.

The invention also provides use of the artificial site-directed mutant of the gene ZmPLA in maize haploid breeding.

The basic principle of the present invention is as follows: for the candidate gene ZmPLA, the target site sequence is designed on the first exon of the gene, and the first exon of the ZmPLA gene is subjected to mutation screening by the method of CRISPR/Cas9 site-directed mutagenesis, to obtain a transgenic mutant in which the function of the ZmPLA gene is lost. After the selfing of the successfully mutated individual plant, a T1 seed was obtained and replanted, and the pollen of the T1 generation plant homozygous mutant and the heterozygous mutant hybridize with two maize hybrids Zhengdan 958 and Jingke 968 and a progeny was obtained. The hybrid progeny is planted in the field, and the maternal haploid was confirmed according to the methods, such as the growth of the individual plant of the progeny in the field, molecular markers and ploidy identification by flow cytometry.

### DESCRIPTION OF THE DRAWINGS

FIG. 1 is a schematic diagram showing the structure of the ZmPLA gene and the design of the target site for CRISPR/Cas9 technique.

FIG. 2 shows the results of CRISPR-mediated ZmPLA gene site-directed mutagenesis and sequencing detected by PCR and Sanger sequencing.

FIG. 3 is a photograph of a haploid appearing after ZmPLA hybridized with the hybrids Zhengdan 958 and Jingke 968.

FIG. 4 shows the results of ploidy identification of haploid leaves in the field.

FIG. 5 shows the results of molecular marker identification of haploids in the field.

### DETAILED DESCRIPTION OF THE INVENTION

The experimental methods used in the following examples are conventional methods unless otherwise specified.

The materials, reagents and the like used in the following examples are commercially available unless otherwise specified.

### Example 1. Method for Inducing Production of Maize Maternal Haploids

I. Localization of Genes Associated with Phenotypes in Maize Maternal Haploids

By locating the QTLs associated with the induction rate in the inducing line derived from the maize maternal haploid Stock6, a major QTL-qhir1 controlling haploid induction was obtained, and functional annotation and candidate gene prediction were performed on the genes in the localization interval and a candidate gene ZmPLA was finally determined.

### II. Maize Maternal Haploid Inducing Ability Obtained by Knocking Out Maize ZmPLA Gene

### 1. Knocking Out Maize ZmPLA Gene Using CRISPR/Cas9 System

1) Selection of sgRNA Sequence

FIG. 1 is a schematic diagram of the gene structure and the target site.

The genomic sequence of the maize ZmPLA gene is shown in SEQ ID NO: 1 in the sequence listing. The sequence of the first exon of the maize ZmPLA gene is shown in SEQ ID NO: 2 in the sequence listing (the sequence of SEQ ID NO: 2 is the sequence of positions 91-450 of SEQ ID NO: 1).

The target site sequence was designed on the first exon sequence of the maize ZmPLA gene and was 21 bp in length, located at positions 264-286 of the first exon.

The target site sequence is GCTGCAGGAGCTGGACGGACCGG (SEQ ID NO: 3).

The designed target site sgRNA sequence is GCUGCAGGAGCUGGACGGACCGG (SEQ ID NO: 4), and the DNA molecule encoding the sgRNA is SEQ ID NO: 3.

### 2) Construction of the CRISPR/Cas9 Vector

The CRISPR/Cas9 vector was obtained by inserting the DNA molecule encoding the sgRNA shown in SEQ ID NO: 3 in the sequence listing into the pBUN411 vector (recorded in the following literature: Xing H L, Dong L, Wang Z P. et al. A CRISPR/Cas9 toolkit for multiplex genome editing in plants[J]. BMC plant biology, 2014, 14(1): 1).

### 3) Acquisition of Transgenic Maize

The CRISPR/Cas9 vector was transferred to Agrobacterium competent cell EHA105 by heat shock transformation to obtain a recombinant EHA 105/CRISPR/Cas9 vector.

Agrobacterium EHA105 competent cells are purchased from HUAYUEYANG BIOTECHNOLOGY CO., LTD., and are commercially available to the public.

The recombinant EHA05/CRISPR/Cas9 vector was transformed to maize Xu178 (recorded in the following literature: Xiang Yan, Wu Daqiang, Jiang Haiyang, et al. Establishment of plant regeneration system from mature embryos of maize elite inbred lines[J]. Acta Laser Biology Sinica, 2007, 16 (5): 649-654, and can be obtained by the public from the National Maize Improvement Center of China Agricultural University) young embryos by Agrobacterium infection method (recombinant Agrobacterium was subjected to 28 Cpropagation, using the bacteria solution after propagation to infect maize young embryos) and then T0 generation transgenic maize plants were obtained after screening, differentiation and rooting.

4) Identification of ZmPLA Gene Transgenic Maize with Mutation

The leaves of the T0 generation transgenic maize plants were collected, and genomic DNA was extracted as a template, and the following primers were used to conduct PCR amplification. PCR amplification products of different lines were obtained.

### ZmPLA Mutation Sequence Detection Primers:

1240F:
CCCUCGACGAGUAUCUAUAGC
1240R:
GAAGAUGAUAGGCUGCAGC.

PCR amplification products of different lines were subjected to Sanger sequencing, and the sequencing results were compared with the first exon of the wild-type maize ZmPLA gene (SEQ ID NO: 2) to identify whether the ZmPLA gene was mutated in different lines of the T0 generation transgenic maize.

The results were as follows: among the 21 plants of T0 generation transgenic maize, the ZmPLA gene was mutated in 8 plants, and the specific mutation forms are as follows, as shown in FIG. 2:

The ZmPLA mutant gene ZmHIR1-1 is a DNA molecule with a sequence obtained by inserting a base T between positions 280 and 281 of the nucleotide sequence of the ZmPLA gene shown in SEQ ID NO: 1;

the ZmPLA mutant gene ZmHIR1-2 is a DNA molecule with a sequence obtained by deleting the 11 bases at positions 271-281 of the nucleotide sequence of the ZmPLA gene shown in SEQ ID NO:1.

The ZmPLA mutant gene ZmHIR1-3 is a DNA molecule with a sequence obtained by deleting the base G at position 281 of the nucleotide sequence of the ZmPLA gene shown in SEQ ID NO: 1; The plant in which the ZmPLA gene mutates was named as positive T0 generation transgenic maize.

5) Genotype Identification of T1 Generation Transgenic Maize with ZmPLA Gene Mutation

The seeds of the positive T0 generation transgenic maize obtained in step 1 were harvested and then sown to obtain T1 generation transgenic maize.

The specific steps for identifying whether the ZmPLA gene of T1 generation transgenic maize is a mutant genotype were as follows: the genomic DNA of the T1 generation transgenic maize was used as a template to conduct amplification using ZmPLA mutation sequence detection primers: 1240F: CCCTCGACGAGTATCTATAGC and 1240R: GAAGATGATAGGCTGCAGC, the PCR products were subjected to Sanger sequencing. According to the sequencing results, the genotypes of the T1 generation transgenic maize were classified.

In the sequencing results, the sequence with bimodal characteristics from the target site sequence indicated a heterozygous genotype, i.e. T1 generation transgenic maize heterozygous ZmPLA gene mutation (ZmPLA gene mutation was present in one of the homologous chromosomes, the ZmPLA gene was not mutated in the other of the homologous chromosomes);

compared with the first exon (SEQ ID NO: 2) of the maize ZmPLA gene, if the sequence with specific unimodal characteristics from the target site sequence was the same, it indicated a wild-type and no mutation occurred, which was not considered in the following analysis; if there was a mutation, it indicated that a homozygous mutation was obtained after the selfing of the T0 generation plant, i.e. T1 generation transgenic maize ZmPLA gene mutation homozygote (the ZmPLA genes in both homologous chromosomes were mutated). The T1 generation transgenic maize heterozygous ZmPLA gene mutant lines were ZmHIR1-1 and ZmHIR-2, and the mutation types of each line were as follows:
one of the homologous chromosomes in the T1 generation transgenic maize ZmPLA gene mutant heterozygous line ZmHIR1-1 contained a ZmPLA mutant gene, and the mutant gene was a DNA molecule with a sequence obtained by inserting a base T between positions 280 and 281 of the nucleotide sequence of the ZmPLA gene (SEQ ID NO: 1) and keeping the other bases unchanged, and the other contained the wild-type ZmPLA gene;
one of the homologous chromosomes in the T1 generation transgenic maize ZmPLA gene mutant heterozygous line ZmHIR1-2 contained a ZmPLA mutant gene, and the mutant gene was a DNA molecule with a sequence obtained by deleting the bases GAGCTGGACGG at positions 271-281 of the nucleotide sequence of the ZmPLA gene (SEQ ID NO: 1) and keeping the other bases unchanged, and the other contained the wild-type ZmPLA gene;
the two homologous chromosomes in the T1 generation transgenic maize ZmPLA gene mutant homozygous line ZmHIR-3 both contained a ZmPLA mutant gene, and the mutant gene was a DNA molecule with a sequence obtained by deleting the base G at position 281 of the nucleotide sequence of the ZmPLA gene (SEQ ID NO: 1) and keeping the other bases unchanged.

### 2. Identification of Haploid Inducing Ability of Mutants Obtained by Knockout of Maize ZmPLA Gene by CRISPR/Cas9 System

### 1) Identification of Haploid Inducing Ability of the T1 Generation Heterozygous Genotype Transgenic Maize ZmPLA Gene Mutant Individual Plant

### (1) Phenotype Identification in Field

The hybrid Zhengdan 958 (Du Chunxin, Cao Chunjing, Cao Qing, et al. The breeding and application of maize hybrid Zhengdan 958 [J]. Journal of Maize Sciences, 2006, 14 (6): 43-45 or obtained from Origin Seed Technology Inc.) and hybrid Jingke 968 (hybrid Jingke 968 was purchased from Beijing Tunyu Seed Industry Co., Ltd., the catalogue number: Tunyu Jingke 968, the public can purchase it from Beijing Tunyu Seed Industry Co., Ltd.) were pollinated with the pollen of the T1 generation transgenic maize ZmPLA gene heterozygous mutant lines ZmHIR1-1 and ZmHIR1-2 respectively to obtain hybrid progeny;

Selfing progeny was obtained by selfing the T1 generation transgenic maize ZmPLA gene heterozygous mutant line ZmHIR1-2.

The above progeny was sown in the field, and the phenotype of the individual plant of the progeny was observed. The haploid had the characteristics of short plant, relative narrow leaves, overshoot, compact plant type, male sterility, etc., and the diploid had the characteristics of tall plant, large and scattered leaves and normal fertility.

The progeny of the wild-type maize (in which the ZmPLA gene was not mutated) and the hybrids was taken as the control. The detection number of each line is shown in Table 1.

The statistical results are shown in Table 1 and FIG. 3:

One out of fifty-four plants of the hybrid progeny of the T1 generation transgenic maize ZmPLA heterozygous gene mutant line ZmHIR1-1 and the hybrid Zhengdan 958 showed haploid traits, which was a quasi-haploid plant;
One out of fifty plants of the hybrid progeny of the T1 generation transgenic maize ZmPLA heterozygous gene mutant line ZmHIR1-1 and the hybrid Jingke 968 showed haploid traits, which was a quasi-haploid plant;
Two out of ninety-three plants of hybrid progeny of the T1 generation transgenic maize ZmPLA heterozygous gene mutant line ZmHIR1-2 and the hybrid Zhengdan 958 showed haploid traits, which were quasi-haploid plants;
Two out of fifty-seven plants of the hybrid progeny of the T1 generation transgenic maize ZmPLA heterozygous gene mutant line ZmHIR1-2 and the hybrid Jingke 968 showed haploid traits, which were quasi-haploid plants;
One out of twenty-seven plants of the selfing progeny of the T1 generation transgenic maize ZmPLA heterozygous gene mutant line ZmHIR1-2 showed haploid traits, which was a quasi-haploid plant.

### (2) Ploidy Identification by Flow Cytometry

The total two plants identified to show the haploid traits in the progeny of the ZmHIR1-1 and the hybrid, the total four plants identified to show the haploid traits in the progeny of the ZmHIR1-2 and the hybrid, and the one plant identified to show the haploid traits in the selfing progeny of the ZmHIR1-2 in above step (1) were subjected to flow cytometry, as follows:

the cell nucleus of the young leaf of the plant to be tested was extracted, and the diploid maize leaf was used as the control; then the signal was detected by flow cytometry, the signal of the diploid cell nucleus was detected first, and the signal peak of the diploid cell nucleus was set to 100 (due to that the genetic material in the diploid cell was twice that of the haploid cell, so the signal peak of the haploid cell nucleus appeared near 50); if the signal peak of the plant to be tested appeared near 100, it was considered that the plant had the same position of the signal intensity enrichment as that of the diploid cell nucleus, and the plant to be tested was diploid. If the signal peak of the cell nucleus of the plant to be tested appeared near 50, the plant to be tested was considered to be a haploid plant.

The detection number of each line is shown in Table 1.

The results are shown in FIG. 4. The top figure shows the result of flow cytometry of the wild-type maize and the bottom figure shows the result of flow cytometry of the T1 generation transgenic maize ZmPLA gene mutant heterozygous line;

the results are as follows:

After the flow cytometry detection, the ploidy of the two quasi-haploid plants identified by the phenotype identification in the hybrid progeny of the ZmHIR1-1 and the hybrids were all found to be haploid, and they were named as T1 generation transgenic maize ZmPLA heterozygous gene mutant line ZmHIR1-1 quasi-haploid plants.

After the flow cytometry detection, the ploidy of the four quasi-haploid plants identified by the phenotype identification in the hybrid progeny of the ZmHIR1-2 and the hybrids were all found to be haploid, and they were named as T1 generation transgenic maize ZmPLA heterozygous gene mutant line ZmHIR1-2 quasi-haploid plants.

After the flow cytometry detection, the ploidy of the one quasi-haploid plant identified by the phenotype identification in the selfing progeny of the ZmHIR1-2 was found to be haploid, and it was named as T1 generation transgenic maize ZmPLA heterozygous gene mutant line ZmHIR1-2 quasi-haploid plant.

### (3) Molecular Marker Identification

30 pairs of molecular markers were randomly designed on the genome, amplification and polymorphic molecular marker screening was conducted using the genomic DNA of the transgenic material Xu178 (Xiang Yan, Wu Daqiang, Jiang Haiyang, et al. Establishment of plant regeneration system from mature embryos of maize elite inbred lines[J]. Acta Laser Biology Sinica, 2007, 16 (5): 649-654, and can be obtained by the public from the National Maize Improvement Center of China Agricultural University) and the hybrids Zhengdan 958 and Jingke 968 as templates. A pair of molecular markers was finally obtained. The PCR product was 500 bp in Xu178, and the length of the product was 300 bp in the hybrid Zhengdan 958 and the hybrid Jingke 968. These two products were quite different and could be identified by agarose gel electrophoresis. The PCR product of Xu178 was larger and its electrophoresis speed was slower, while the PCR product fragment of the hybrid Zhengdan 958 and the hybrid Jingke 968 was smaller and its electrophoresis speed was faster. Therefore, the band of Xu178 was located above the bands of the hybrid Zhengdan 958 and the hybrid Jingke 968. (FIG. 5, lanes 3 and 4 are the banding patterns of the hybrid Zhengdan 958 and the hybrid Jingke 968 respectively, and lane 5 is the banding pattern of Xu178)

The two quasi-haploid plants in the hybrid progeny of the T1 generation heterozygous gene mutant line ZmHIR1-1 and the hybrids and the four quasi-haploid plants in the hybrid progeny of the T1 generation heterozygous gene mutant line ZmHIR1-2 and the hybrids were subjected to genomic DNA extraction, PCR and agarose banding pattern detection. If the plant to be tested only had the band of Zhengdan 958 (FIG. 5, lane 1), it was considered that the individual plant had no banding pattern of the paternal plant, therefore was a maternal haploid. If the bands of Xu178 and Zhengdan 958/Jingke 968 were simultaneously present in the individual plant of the hybrid progeny (FIG. 5, lane 2), it was considered that the individual plant was progeny of normal hybrid and was a diploid.

The results are shown in FIG. 5, M: Marker, 5 is the banding pattern of paternal Xu178, 4 is the banding pattern of maternal Zhengdan 958, 3 is the banding pattern of maternal Jingke 968, 1 is the banding pattern of a haploid in the progeny, 2 is the banding pattern of a heterozygous diploid in the progeny.

The molecular marker identification results are as follows:

The molecular marker identification results of the two quasi-haploid plants identified by the phenotype identification in the progeny of the ZmHIR1-1 and the hybrids showed that they were both maternal haploid plants.

The molecular marker identification results of the four quasi-haploid plants identified by the phenotype identification in the progeny of ZmHIR1-2 and the hybrids showed that they were all maternal haploid plants.

Therefore, if the individual plant in the progeny of the heterozygous transgenic line and the hybrid or the individual plant in the selfing progeny of the heterozygous transgenic line is identified as a haploid according to any one of the identification results of the above three methods, the plant is a maize maternal haploid or maize maternal haploid candidate; if none of the identification results of the above three methods show the plant is a haploid, it is neither a maize maternal haploid nor a maize maternal haploid candidate.

The above identification results are shown in Table 1. Haploid induction rate (%)=(number of haploid plants/total number of test plants)*100. It can be seen that a maize maternal haploid is available in the hybrid progeny of the material in which the ZmPLA gene is mutated and other materials.

TABLE 1
The frequency of occurrence of haploid plants in
the progeny of the test heterozygous mutant lines

| | | Total | number of | |
|---|---|---|---|---|
| | | number of | haploid | induction |
| Line | Test material | test plants | plants | rate (%) |
| ZmHIR1-1 | Zhengdan958 | 54 | 1 | 1.85 |
| ZmHIR1-1 | Jingke968 | 50 | 1 | 1.92 |
| ZmHIR1-2 | Zhengdan 958 | 93 | 2 | 0.22 |
| ZmHIR1-2 | Jingke 968 | 57 | 2 | 1.90 |
| ZmHIR1-2 | selfing | 27 | 1 | 3.70 |
| Control | Zhengdan 958 | 306 | 0 | 0 |
| Control | Jingke 968 | 187 | 0 | 0 |

Note:
The control is the progeny obtained after the hybrids Zhengdan 958 and Jingke 968 were pollinated with the pollen of wild-type Xu178 material.
2) Identification of haploid inducing ability of T1 generation homozygous genotype transgenic maize ZmPLA gene mutant individual plant
(1) Phenotype identification in field The hybrid Zhengdan 958 was pollinated with the pollen of the T1 generation transgenic maize ZmPLA gene homozygous mutant line ZmHIR1-3 to obtain hybrid progeny; selfing progeny was obtained by selfing the T1 generation transgenic maize ZmPLA gene heterozygous mutant line ZmHIR1-3.

The above progeny was sown in the field, and the phenotype of the individual plant of the progeny was observed. The haploid had the characteristics of short plant, relative narrow leaves, overshoot, compact plant type, male sterility, etc., and the diploid had the characteristics of tall plant, large and scattered leaves and normal fertility.

The results are as follows:

Four out of two hundred and fifty-six plants of the hybrid progeny of the T1 generation transgenic maize ZmPLA homozygous gene mutant line ZmHIR1-3 and the hybrid Zhengdan 958 showed haploid traits, which were quasi-haploid plants; Two out of thirty plants of the selfing progeny of the T1 generation transgenic maize ZmPLA homozygous gene mutant line ZmHIR1-3 showed haploid traits, which were quasi-haploid plants.

### (2) Ploidy Identification by Flow Cytometry

The four quasi-haploid plants in the hybrid progeny of T1 generation transgenic maize ZmPLA homozygous gene mutant line ZmHIR1-3 and the hybrid Zhengdan 958 and the two quasi-haploid plants in the selfing progeny of the ZmHIR-3 homozygous mutant were subjected to flow cytometry, as follows:

the cell nucleus of the young leaf of the plant to be tested was extracted, and the wild-type maize (ZmPLA gene was not mutated, diploid) leaf was used as the control; then the signal was detected by flow cytometry, the signal of the diploid cell nucleus was detected first, and the signal peak of the diploid cell nucleus was set to 100 (due to that the genetic material in the diploid cell was twice that of the haploid cell, so the signal peak of the haploid cell nucleus appeared near 50); if the signal peak of the plant to be tested appeared near 100, it was considered that the plant had the same position of the signal intensity enrichment as that of the diploid cell nucleus, and the plant to be tested was diploid. The detection number of each line is shown in Table 2.

The results are shown in FIG. 4. The top figure shows the detection result of flow cytometry of the wild-type maize and the bottom figure shows the detection result of flow cytometry of the quasi-haploid plants in the progeny of the T1 generation transgenic maize ZmHIR1-3 homozygous line;

the results are as follows:

After the flow cytometry detection, the ploidy of the four quasi-haploid plants in the hybrid progeny of the ZmHIR1-3 and Zhengdan 958 were all found to be haploid.

After the flow cytometry detection, the ploidy of the two quasi-haploid plants in the selfing progeny of the ZmHIR1-3 homozygous mutant material were all found to be haploid.

### (3) Molecular Marker Identification

30 pairs of agarose molecular markers were randomly designed on the genome, amplification and polymorphic molecular marker screening was conducted using the genomic DNA of the transgenic material Xu178 and the hybrids Zhengdan 958 and Jingke 968 as templates. A pair of molecular markers was finally obtained. The PCR product was 500 bp in Xu178, and the length of the product was 300 bp in the hybrid Zhengdan 958 and the hybrid Jingke 968. These two products were quite different and could be identified by agarose gel electrophoresis. The PCR product of Xu178 was larger and its electrophoresis speed was slower, while the PCR product fragment of the hybrid Zhengdan 958 and the hybrid Jingke 968 was smaller and its electrophoresis speed was faster. Therefore, the band of Xu178 was located above the bands of the hybrid Zhengdan 958 and the hybrid Jingke 968. (FIG. 5, lanes 3 and 4 are the banding patterns the hybrid Zhengdan 958 and the hybrid Jingke 968 respectively, and lane 5 is the banding pattern of Xu178)

The four quasi-haploid plants in the hybrid progeny of the ZmHIR1-3 T1 generation transgenic maize homozygous gene mutant line and Zhengdan 958 selected in the field were subjected to genomic DNA extraction, PCR and agarose banding pattern detection. If the plant to be tested only had the band of Zhengdan 958 (FIG. 5, lane 1), it was considered that individual plant had no banding pattern of the paternal plant, therefore was a maternal haploid. If the bands of Xu178 and Zhengdan 958 were simultaneously present in the individual plant of the hybrid progeny (FIG. 5, lane 2), it was considered that the individual plant was progeny of normal hybrid and was a diploid.

The results are shown in FIG. 5, M: Marker, 5 is the banding pattern of Xu178, 4 is the banding pattern of the hybrid Zhengdan 958, 3 is the banding pattern of the hybrid Jingke 968, 1 is the banding pattern of a haploid in the progeny, 2 is the banding pattern of a homozygous diploid in the progeny.

The results are as follows:

After the four quasi-haploid plants in the hybrid progeny of the T1 generation transgenic maize ZmHIR-3 homozygous gene mutant line and Zhengdan 958 were detected by the molecular marker identification, they all found to be maternal haploid plants.

Therefore, if the individual plant in the progeny of the homozygous transgenic line and the hybrid or the plant in the selfing progeny of the homozygous transgenic line is identified as a haploid according to any one of the identification results of the above three methods, the plant is a maize maternal haploid or a maize maternal haploid candidate; if none of the identification results of the above three methods show the plant is a haploid, it is neither a maize maternal haploid nor a maize maternal haploid candidate.

The results are shown in Table 2. Induction rate (%)=(number of haploid plants/total number of test plants)*100. It can be seen that a maize maternal haploid is available in the hybrid progeny of the material in which the ZmPLA gene is mutated and other materials.

TABLE 2
The frequency of occurrence of haploid plants in
the progeny of the test heterozygous mutant lines

| | | Total | Number | |
|---|---|---|---|---|
| | | number of | of haploid | Induction |
| Line | Test material | test plants | plants | rate(%) |
| ZmHIR1-3 | ZhengDan 958 | 256 | 4 | 1.54 |
| ZmHIR1-3 | Selfing | 30 | 2 | 6.67 |
| Control | ZhengDan 958 | 406 | 0 | 0 |

### III. Genotype Identification of the Maize Maternal Haploid Stock6

Stock6 is the first reported special material that can induce the production of maize maternal haploid (Coe E H (1959) Aline of maize with high haploid frequency. Am Nat 93:381-382). After fine mapping of the major QTLs for induction rate and prediction of candidate genes, it was found that there were multiple SNP mutations and a 4 bp insertion in the ZmPLA gene of Stock6 compared to B73 (Table 3), which makes the normal function of the gene lost. Using the CRISPR technique to carry outsite-directed mutagenesis of the ZmPLA gene of wild-type maize material, it was proved that a certain frequency of haploid could appear in the progeny obtained after other materials were pollinated by the paternal material in which the gene was mutated. The ZmPLA gene in the Stock6 genome is a mutant sequence obtained by mutating the gene ZmPLA shown in SEQ ID NO: 1 as follows and named ZmHIR-Stock6.

TABLE 3
Mutation types of the exons of the gene ZmPLA

| | |
|---|---|
| Position | Nucleotide |

| (from 5′UTR) | Type | ZmPLA | ZmHIR-Stock 6 |
|---|---|---|---|
| 409 | SNP | C | T |
| 421 | SNP | C | G |
| 441 | SNP | T | C |
| 887 | SNP | T | G |
| 1210 | SNP | G | C |
| 1306 | SNP | T | C |
| 1435 | SNP | G | A |
| 1471 | SNP | C | A |
| 1541 | SNP | A | C |
| 1569 | Insertion | | CGAG |
| 1588 | SNP | T | C |
| 1591 | SNP | C | A |
| 1687 | SNP | A | C |
| 1691 | SNP | G | A |
| 1706 | SNP | T | C |
| 1708 | SNP | G | C |

The 5′UTR region of the gene ZmPLA is mutated as follows: two bases TA are deleted at positions 45-46, the bases TCG at positions 65-67 are substituted with CAA, two bases TC are inserted between positions 67 and 68 and the bases TT at positions 80-81 are substituted with CG.

The intron region of the gene ZmPLA is mutated as follows: the bases GTAC at positions 499-503 are deleted, the base C at position 524 was mutated to G, the base G at position 530 was mutated to T, the bases GCATGCAT at positions 553-560 are deleted and the bases GTAC at positions 806-809 are deleted.

The 3′UTR region of the gene ZmPLA is mutated as follows: the base G at position 1741 was mutated to A, the base C at position 1781 is mutated to T and the base A at position 1787 is mutated to T.

The specific mutation types of the SNP and Insertion mutations of the ZmPLA mutant gene 4 in the above-mentioned inducing line Stock 6 compared to wild-type ZmPLA gene in B73 are as follows:

the ZmHIR-Stock6 mutant sequence is a DNA molecule with a sequence obtained by inserting CGAG after position 1569 of the nucleotide sequence SEQ ID NO:1 of the ZmPLA gene, and mutating the base C at position 409 to a base T, mutating the base C at position 421 to a base G, mutating the base T at position 441 to a base C, mutating the base T at position 887 to a base G, mutating the base G at position 1210 to a base C, mutating the base T at position 1306 to a base C, mutating the base G at position 1435 to a base A, mutating the base C at position 1471 to a base A, mutating the base A at position 1541 to a base C, mutating the base T at position 1588 to a base C and mutating the base C at position 1591 to a base A, mutating the base A at position 1687 to a base C, mutating the base G at position 1691 to a base A, mutating the base T at position 1706 to a base C, mutating the base G at position 1708 to a base C, deleting the two bases TA at positions 45-46, substituting the bases TCG at positions 65-67 with bases CAA, inserting two bases TC between positions 67 and 68, substituting the bases TT at positions 80-81 with bases CG, deleting the bases GTAC at positions 499-503, mutating the base C at position 524 to a base G, mutating the base G at position 530 to a base T, deleting the bases GCATGCAT at positions 553-560, deleting the bases GTAC at positions 806-809, mutating the base G at position 1741 to a base A, mutating the base C at position 1781 to a base T, mutating the base A at position 1787 to a base T.

The above bases CGAG insertion after position 1482 results in a frameshift mutation in the gene. The SNP mutations at positions 319, 331 and 1120 result in amino acid changes and also affect protein function.

In the progeny of the maize maternal haploid Stock6, haploid was confirmed by all of the haploid traits, leaf ploidy identification by flow cytometry and molecular marker identification.

Therefore, no matter which mutation of maize ZmPLA gene causes the loss of the function, it can result in a maize maternal haploid

### INDUSTRIAL APPLICATION

The experiments of the present invention prove that the mutation of ZmPLA can result in the production of a maize maternal haploid, which lays an important foundation for revealing the genetic and biological mechanisms of the production of a maize maternal haploid. At the same time, the mutant plants obtained by the experiments or the methods of the present invention have the maize maternal haploid inducing ability, which is important for selecting a new inducing line, further increasing the induction rate and improving the efficiency of the maize haploid breeding.

What is claimed is:

1. A ZmPLA mutant gene, having a nucleotide sequence which is obtained by conducting insertion or/and deletion or/and substitution mutations in the nucleotide sequence of the wild-type ZmPLA gene;
the nucleotide sequence of the wild-type ZmPLA gene is SEQ ID NO: 1.

2. The ZmPLA mutant gene according to claim 1, wherein the nucleotide sequence of the ZmPLA mutant gene is any one of the following 1) to 4):
1) a sequence obtained by inserting a base T between positions 280 and 281 of the nucleotide sequence of the wild-type ZmPLA gene and keeping the other bases unchanged;
2) a sequence obtained by deleting the bases at positions 271-281 of the nucleotide sequence of the wild-type ZmPLA gene and keeping the other bases unchanged;
3) a sequence obtained by deleting the base G at position 2810f the nucleotide sequence of the wild-type ZmPLA gene and keeping the other bases unchanged;
4) a DNA molecule with a sequence obtained by inserting CGAG after position 1569 of the nucleotide sequence of the wild-type ZmPLA gene, and mutating the base C at position 409 to a base T, mutating the base C at position 421 to a base G, mutating the base T at position 441 to a base C, mutating the base T at position 887 to a base G₉ mutating the base G at position 1210 to a base C, mutating the base T at position 1306 to a base C, mutating the base G at position 1435 to a base A, mutating the base C at position 1471 to a base A, mutating the base A at position 1541 to a base C, mutating the base T at position 1588 to a base C and mutating the base C at position 1591 to a base A, mutating the base A at position 1687 to a base C, mutating the base G at position 1691 to a base A, mutating the base T at position 1706 to a base C, mutating the base G at position 1708 to a base C, deleting the two bases TA at positions 45-46, substituting the bases TCG at positions 65-67 with bases CAA, inserting two bases TC between positions 67 and 68, substituting the bases TT at positions 80-81 with bases CG, deleting the bases GTAC at positions 499-503, mutating the base C at position 524 to a base G, mutating the base G at position 530 to a base T, deleting the bases GCATGCAT at positions 553-560, deleting the bases GTAC at positions 806-809, mutating the base G at position 1741 to a base A, mutating the base C at position 1781 to a base T, mutating the base A at position 1787 to a base T and keeping the other bases unchanged.

3. Use of the mutant gene according to claim 1 or the nucleotide sequence of the wild-type ZmPLA gene for producing haploid or in double haploid (DH) breeding.

4. Use of silencing or inhibiting the expression of the ZmPLA gene or knocking out the ZmPLA gene in the genome of a target plant in the production of a plant haploid; or use of a substance which silences or inhibits the expression of the ZmPLA gene or knocks out the ZmPLA gene in the genome of a target plant in the production of a plant maternal haploid.

5. The use according to claim 4, wherein the silencing or inhibiting the expression of the ZmPLA gene or knocking out the ZmPLA gene in the genome of the target plant is such that the expression level of the ZmPLA gene in the genome of the target plant is decreased or a deletion or insertion mutation occurs.

6. The use according to claim 5, wherein the deletion or insertion mutation in the ZmPLA gene in the genome of the target plant is the deletion or insertion mutation in the first exon and/or the second exon and/or the third exon and/or the fourth exon of the ZmPLA gene in the genome of the target plant; or
a way to cause the deletion or insertion mutation in the ZmPLA gene in the genome of the target plant is CRISPR/Cas9 and/or TALEN technology and/or T-DNA insertion and/or EMS mutagenesis.

7. The use according to claim 6, wherein the way to cause the deletion or insertion mutation in the first exon of the ZmPLA gene in the genome of the target plant is CRISPR/Cas9; or
the substance which silences or inhibits the expression of the ZmPLA gene or knocks out the ZmPLA gene in the genome of the target plant is a substance which causes deletion or insertion mutation of the first exon of the ZmPLA gene in the genome of the target plant;
the substance which causes deletion or insertion mutation of the first exon of the ZmPLA gene in the genome of the target plant is CRISPR/Cas9 system;
the target sequence of the CRISPR/Cas9 system is positions 264-276 of the first exon and shown in SEQ 1:1) NO: 3;
the sgRNA sequence of the CRISPR/Cas9 system is SEQ ID NO: 4.

8. Use of silencing or inhibiting the expression of the ZmPLA gene or knocking out the ZmPLA gene in the genome of a target plant in double haploid line breeding or line-based hybrid breeding; or use of a substance which silences or inhibits the expression of the ZmPLA gene or knocks out the ZmPLA gene in the genome of a target plant in double haploid breeding or DH line-based hybrid breeding.

9. A substance which silences or inhibits the expression of the ZmPLA gene or knocks out the ZmPLA gene in the genome of a target plant, comprising CRISPR/Cas9 system, the target sequence of the CRISPR/Cas9 system is positions 264-286 of the first exon and shown in SEQ ID NO: 3; the sgRNA sequence of the CRISPR/Cas9 system is the SEQ ID NO: 4.

10. The use according to claim 3, wherein the plants are corn or other plants.

11. The substance according to claim 9, wherein the plants are corn or other plants.


SEQUENCE LISTING (4 sequences)

SEQ ID NO: 1 - DNA, 1795 bp, Artificial Sequence
  Synthesized
agttcatcactaatcacacttattgtgccctcgacgagtatctatagctagctcattaatcgattcgggggtgtgttgtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctggaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatgcagaactgcccgcgcatctttcctcagaagtgagtccgatgctgccgccattgttcttgcatccatccagcatcgtacgtacgtcctctatacatctgcggatcatcatgtgcgcatgtttgtggcatgcatgcatgcatgtgagcaggagcaggcttgcggccgccatgtccgcgctgaggaagccaaagtacaacggcaagtgcatgcgcagcctgattaggagcatcctcggcgagacgagggtaagcgagacgctgaccaacgtcatcatccctgccttcgacatcaggctgctgcagcctatcatcttctctacctacgacgtacgtacgtcgtcacgaatgattcatctgtacgtcgtcgcatgcgaatggctgcctacgtacgccgtgcgctaacatactcagctctttcctatctgctgcgccaatttgcaggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccggtaactgactagctaactggaaaacggacgcacagactccatgtccatggcggcccacaaggtcgatgctaattgttgcttatgtatgtcgcccgattgcacatgcgtagacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggagaacaaggctcgcgcgccgcgtctctgccatcaacccaagaggctctagatgtgcgtcgtacgatatctaagacaagtggctttactgtcagtcacatgcttgtaaataagtagactttattttaataaaacataaaaatatatat

SEQ ID NO: 2 - DNA, 360 bp, Artificial Sequence
  Synthesized
atggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctggaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatgcagaactgcccgcgcatctttcctcagaag

SEQ ID NO: 3 - DNA, 23 bp, Artificial Sequence
  Synthesized
gctgcaggagctggacggaccgg

SEQ ID NO: 4 - RNA, 23 nt, Artificial Sequence
  Synthesized
gcugcaggagcuggacggaccgg